(19) United States
(12) Patent Application Publication
(10) Pub. No.: US 2007/0148746 A1
(43) Pub. Date: Jun. 28, 2007

(54) PROCESS FOR THE ENZYMATIC SYNTHESIS OF TRIGLYCERIDES

(76) Inventors: ULRICH SCHOERKEN, Duesseldorf (DE); Carolin Meyer, Duesseldorf (DE); Peter Horlacher, Bellenberg (DE); Sabine Both, Neuss (DE)

Correspondence Address: COGNIS CORPORATION;PATENT DEPARTMENT, 300 BROOKSIDE AVENUE, AMBLER, PA, 19002, US

(21) Appl. No.: 11/565,696

(22) Filed: Dec. 1, 2006

(30) Foreign Application Priority Data
Dec. 3, 2005 (DE) 10 2005 057 832.2

(51) Int. Cl. C12P 7/64; C07C 57/02

(52) U.S. Cl. 435/134; 554/1

(57) ABSTRACT

The invention relates to a process for the enzymatic synthesis of esters of polyunsaturated, short-chain or sensitive fatty acids with a high triglyceride content, which is made up of an enzymatic synthesis of the mixture of triglycerides and partial glycerides starting from free fatty acid, methyl or ethyl esters and glycerol, a subsequent selective enzymatic back-hydrolysis of the partial glycerides to fatty acid and glycerol and distillation-based separation of the triglycerides, and to the triglyceride-rich ester mixtures obtainable by this process. The process is preferably carried out as a one-pot process in the same reactor and in the presence of the same enzyme.

### RELATED APPLICATIONS

This application claims priority from German Application Serial No. 10 2005 057 832.2 filed Dec. 3, 2005, the entire contents of which are incorporated herein by reference.

### FIELD OF THE INVENTION

This invention relates generally to fatty acid esters and, more particularly, to a new process for the enzymatic synthesis of esters of polyunsaturated, short-chain or sensitive fatty acids with a high triglyceride content, which is made up of an enzymatic synthesis of the mixture of triglycerides and partial glycerides starting from free fatty acid or alkyl esters thereof and glycerol, a subsequent selective enzymatic back-hydrolysis of the partial glycerides to fatty acid and glycerol and distillation-based separation of the triglycerides from fatty acids and/or excess alkyl esters and glycerol, and to the triglyceride-rich ester mixtures obtainable by this process.

### BACKGROUND OF THE INVENTION

The chemical synthesis of glyceride esters of polyunsaturated fatty acids and sensitive fatty acids has the disadvantage that very high temperatures and large quantities of chemical catalysts generally have to be used, so that secondary products and unwanted isomerizations occur to a relatively high degree. Enzyme-catalyzed reactions with lipases generally take place under milder conditions and give high-purity end products. Saturated fatty acids and alkyl esters thereof, such as for example medium-chain fatty acids (more particularly C8 and C10), which are obtained from palm kernel oil and coconut oil, release color and odor in high-temperature syntheses. Fatty acids and esters obtained in particular from coconut oil tend to be colored. In this case, too, enzymatic synthesis under mild conditions can lead to products with improved color and odor properties.

European patent application EP 1 322 776 A1 describes a lipase-catalyzed method for the production of triglycerides of polyunsaturated conjugated fatty acids starting from the alkyl ester of the unsaturated fatty acids and glycerol, in which the alcohol formed is removed from the reaction under reduced pressure. International patent application WO 9116443 A1 describes the esterification of glycerol and free polyunsaturated fatty acids or alkyl esters thereof to the corresponding triglycerides by removing the water of reaction or the alcohol formed under reduced pressure.

Unfortunately, enzymatic syntheses often have the disadvantage that the reactions are relatively slow. In particular, an increase in the triglyceride content from, for example, 90% to 95% or even higher is very time-consuming and hence expensive because the reaction rate of the enzymatic synthesis follows the Michaelis-Menten kinetics and is proportional to the concentration of the educts and products. One way of increasing the triglyceride content without significantly lengthening the reaction time is to separate the fatty acids and partial glycerides from the triglycerides by distillation. However, it has been found that the separation of diglycerides is barely possible because very high distillation temperatures are required and lead to a reduction in the product quality of the triglycerides.

Accordingly, the problem addressed by the present invention was to provide an improved process for the production of esters of polyunsaturated, short-chain or sensitive, more particularly polyunsaturated, fatty acids with a high triglyceride content in high yields and short reaction times.

### BRIEF DESCRIPTION OF THE INVENTION

The present invention relates to a process for the enzyme-catalyzed synthesis of esters of polyunsaturated fatty acids, short-chain and/or sensitive fatty acids with a high triglyceride content, in which
(a) a synthesis of the fatty acids or alkyl esters thereof with glycerol to form their triglycerides is carried out in vacuo in the presence of an enzyme under low-water conditions,
(b) the partial glycerides also formed in step (a) are then back-hydrolyzed under normal pressure in the presence of an enzyme after the addition of water, and
(c) fatty acids and/or excess alkyl esters and glycerol are separated from the triglyceride by distillation or refining.

### DETAILED DESCRIPTION OF THE INVENTION

The synthesis and the subsequent selective back-hydrolysis of the partial glycerides are generally carried out in the same reactor by a one-pot process, preferably in the presence of the same enzyme. The enzymatic synthesis of fatty acid glyceride esters (step (a)) with a high triglyceride content generally requires long reaction times which are a problem to sensitive fatty acids. It has now surprisingly been found that the triglyceride content can be increased by carrying out a selective back-hydrolysis (step (b)) of the partial glycerides formed in step (a) immediately after step (a) because the partial glycerides back-hydrolyze more easily than the triglycerides. The ester mixtures thus produced are distinguished by high purity, hardly any secondary products and high stability because the sensitive fatty acids are not damaged by the minimal load and reduced reaction time. In this way, chemically high-quality ester mixtures with a triglyceride content of at least 90%, preferably of at least 95% and even of at least 98%, based on the total glycerides, can be synthesized in a short reaction time of 2 to 25 hours. The process according to the invention is particularly suitable for the synthesis of ester mixtures of unsaturated fatty acids with a high triglyceride content, for the synthesis of MCT oils and for the synthesis of structured lipids.

Advantages of triglyceride-rich esters over 1,3-diglycerides using active fatty acids (CLA, PUFAs):

Most of the lipases suitable for the synthesis of triglycerides, such as Candida antarctica B lipase or Rhizomucor miehei lipase for example, have a strong preference for the 1- and 3-positions of the glycerol. Accordingly, the synthesis proceeds mainly via 1-monoglyceride and 1,3-diglyceride as intermediates. 2-Monoglyceride and 1,2-diglyceride are formed solely by acyl migration and secondary reaction and are quickly further synthesized to the triglyceride. Accordingly, on completion of the synthesis reaction, 1-mono-and 1,3-diglyceride are the main secondary components in the product. The fatty acids bound to the 1- and 3-positions of the glyceride are metabolized differently from the fatty acids bound in the 2-position. The 1- and 3-bound fatty acids are eliminated in the intestine, the 2-monoglyceride remaining intact. The fatty acids and the 2-monoglyceride are adsorbed and triglyceride is resynthesized from 2-monoglyceride and fatty acids. Some of the fatty acids are otherwise metabolized; for example they are transported through the bloodstream and degraded by oxidation to produce energy.

The principle of the different degradation and transport of 2-bound and 1,3-bound fatty acids forms the basis, for example, of the dietetic effect of enova oil, a synthetic diglyceride with a high 1,3-diglyceride content where very little 2-monoglyceride is available for the resynthesis of triglyceride. Since very little resynthesis occurs, fewer fats are incorporated in the fatty cells, instead a relatively high proportion of the fatty acids taken up is converted into energy.

Where triglycerides containing pharmacologically active fatty acids, such as CLA (conjugated linoleic acid), EPA (eicosapentaenoic acid) or DHA (docosahexaenoic acid), are taken up, burning by oxidation leads to a reduction in the dose-related effect. Accordingly, for the reasons explained above, 1,3-diglycerides are a less suitable form for administering these fatty acids, triglycerides being preferred. Because of this, a reduction in the 1,3-diglyceride content by enzymatic selective back-hydrolysis into triglycerides containing pharmacologically active fatty acids is recommended, particularly if no other fat-containing foods are ingested.

In the case of CLA as the active fatty acid, a difference in activity between free fatty acid and triglyceride was demonstrated in a long-term study (Gaullier et al., 2004; AJCN 79; 1118-1125). The administration of triglyceride led after 12 months to an improved reduction in BFM (body fat mass) by comparison with the free acid. The effect of free acid should be comparable with the effect of a 1,3-diglyceride because the 1,3-diglyceride is split completely into glycerol and the free acids in the intestine. Accordingly, it may be indirectly inferred from this study that a CLA glyceride has a particularly good effect if the triglyceride content is very high and the 1,3-diglyceride content is low.

Application for Achieving High OH Value Specifications in Myritols where Direct Synthesis is Difficult:

MCTs (medium-chain triglycerides) are triglycerides which contain caprylic acid and capric acid as their principal components. Caproic acid or lauric acid are bound in small quantities. They are used as an emollient in cosmetic preparations or as a medical food in patients with metabolic disorders. Both applications involve stringent requirements in regard to odor, color and free hydroxyl group content. To satisfy these requirements, purification steps have to be carried out after a chemical synthesis in order to meet above all the odor and color requirements.

With an enzymatic synthesis, products of better quality can be produced at low temperatures. However, it is difficult to meet the requirements concerning the free hydroxyl group content in a direct synthesis. For example, the maximum OH value specified for Myritol (an MCT from Cognis) is 5. In this case, the OH value can be improved by selective back-hydrolysis of mono- and diglycerides bearing free OH groups. Free glycerol and fatty acid are easy to separate from the MCT.

Application to Structured Lipids:

In recent years, there has been a growing number of studies in the literature on the subject of structured lipids which generally consist of mixtures of medium-chain and essential fatty acids. The process described herein is also suitable for increasing the triglyceride content in these structured lipids because each of the individual components can be recognized by the enzymes, so that the mono- and diglycerides can be selectively hydrolyzed from these lipids also. The partial glycerides present in particular in structured lipids containing medium-chain fatty acids should be completely removed because the smoke points of medium-chain partial glycerides in particular are very low. Accordingly, structured lipids with high levels of partial glycerides are unsuitable for roasting and frying.

1. Step (a)-Enzymatic Synthesis of the Mixture of Triglycerides and Partial Glycerides:

The first step of the one-pot process, the synthesis of the triglycerides, is carried out under water-free conditions. Where free fatty acids are selected as the starting material, it has proved to be useful to add salts, preferably sodium carbonate, to accelerate the synthesis. The salts may be added to the reaction mixture either in dry form or dissolved in water, so that—in the later case—very small quantities of water, i.e. less than 1% and preferably less than 0.5%, based on the mixture as a whole, are exceptionally added to the reaction mixture. The synthesis is carried out in vacuo to remove the water of reaction formed where free acids are used or to remove the alcohol formed where the corresponding esters, preferably the methyl or ethyl esters, are used. The reaction equilibrium is thus shifted towards the glyceride synthesis.

The process is applicable to all fatty acids or their alkyl esters, more particularly their methyl or ethyl esters, but is particularly suitable for polyunsaturated fatty acids and polyunsaturated conjugated fatty acids and also conjugated linoleic and linolenic acids. Docosahexaenoic acid, eicosapentaenoic acid, arachidonic acid, gamma-linolenic acid and conjugated linoleic acid, more particularly the c9,t11 and t10,c12 isomers of conjugated linoleic acid (CLA), are preferably used.

To produce MCT oils with low OH values, short-chain fatty acids or methyl esters thereof are typically used.

Typical, but not exclusive, examples of suitable enzymes are lipases, phospholipases and/or esterases of microorganisms selected from the group consisting of Alcaligenes, Aspergillus, Candida, Chromobacterium, Rhizomucor, Penicilium, Pseudomonas, Rhizopus, Thermomyces, Geotrichum, Mucor, Burkholderia and mixtures thereof. Lipases and esterases from such organisms as Alcaligenes, Candida, Chromobacterium, Penicilium, Pseudomonas, Rhizopus, Rhizomucor and Thermomyces are preferred because they are particularly active, lipases from Candida and Rhizomucor, especially the Candida antarctica B lipase, being particularly preferred. The lipases are preferably immobilized on a carrier material, 3 to 12% by weight immobilizate, based on the percentage fat content, being particularly preferred. Various carrier materials suitable for binding enzymes are suitable for the process according to the invention. Suitable carriers include plastics, mineral carriers or resins which bind the esterases via hydrophobic interactions, such as for example Amberlite 16 (Rohm & Haas), Celite or Accurel MP 1000 (Membrana). Other suitable carriers are ion exchangers which bind the esterases through ionic and, in part, hydrophobic interactions, such as for example Dowex Marathon WBA (Dow Chemicals) or Duolite A 568 (Rohm & Haas). Carriers capable of binding the esterases through chemically reactive groups, such as Eupergit (Degussa) for example, may also be used.

Chemical modifications for adapting the esterases to the reaction system are also suitable. Hydrophobic modifications, for example coating with surfactants, or chemical modification with fatty aldehydes may be used. Stabilizing the esterases by crosslinking, for example with glutaraldehyde, DMA or EDC, is also suitable.

Combinations of chemical modification and immobilization for adapting the esterases to the reaction system are also suitable. Either the esterases may first be immobilized and then modified fixed to a carrier or already chemically modified esterases are immobilized.

The temperature range suitable for the reaction is determined by the activity optimum of the enzymes. A temperature range of 40 to 90° C. has proved to be particularly suitable for the lipases preferably used, the range from 60 to 80° C. being particularly preferred. A vacuum of at least 200 mbar, preferably 1 to 100 mbar and more particularly 5 to 60 mbar should be applied. The preferred test parameters will be derived from the increase to be achieved in the reaction rate.

2. Step (b)-Selective Hydrolysis of the Partial Glycerides:

After the enzymatic synthesis under low-water conditions, the partial glycerides formed are hydrolyzed into alcohol and the free acid at relatively low temperatures in a water-rich medium and in the same reaction vessel. By “water-rich” is meant a quantity of water of at most 50%, preferably at most 25% and more particularly at most 20% and more than 1%, preferably at least 2% and more particularly at least 5% water, based on the mixture as a whole.

Enzymes suitable for this purpose are lipases, preferably Candida antarctica B lipase, Penicilium lipases, Thermomyces lipase, porcine pancreas lipases, Rhizomucor lipases; Candida antarctica B lipase and Penicilium camembertii lipase being particularly preferred. The lipases may be directly used as a liquid or powder preparation for the selective hydrolysis and as immobilized enzymes.

The selective back-hydrolysis is preferably carried out in the presence of the same enzyme used in the preceding synthesis. Candida B lipase is particularly suitable. A suitable temperature range for the selective back-hydrolysis is generally 20 to 60° C. and preferably 20 to 45° C. It is thus lower than the synthesis temperature range which is normally 10 to 80° C. and preferably 30 to 50° C. higher than the temperature range of the second process step. The hydrolysis takes place with stirring under normal pressure. Where immobilized enzyme is used, it is removed by filtration after the hydrolysis.

3. Step (c)-Separation of the Triglycerides From Fatty Acids and Glycerol by Distillation

Purification is carried out by removing the aqueous phase using a centrifuge or separator, optionally after slight acidification for better phase separation and to neutralize alkaline additives used in the synthesis. In an alternative process, the water added is removed from the product mixture by distillation.

A distillation of fatty acid and/or excess fatty acid alkyl ester and any monoglyceride still present is then carried out in vacuo, so that the bottom product contains the enriched triglycerides. The fatty acids and/or fatty acid alkyl esters removed and any free glycerol or monoglycerides still present may then be re-used for the triglyceride synthesis.

### EXAMPLES

### Example 1

### Enzyme Screening for the Selective Hydrolysis of Diglycerides in a Commercially Obtainable CLA-TG Preparation (Tonalin TG 80)

13 reaction vessels were each filled with 20 g Tonalin TG 80 and 2 g water. A commercially obtainable enzyme preparation, as indicated in Table 1 below, was added to each mixture. The mixtures were incubated with stirring at room temperature. Samples were taken after 1.5 h and 5 h, the oil phase was removed by centrifuging and the distribution of the glycerides was analyzed by gas chromatography. The result is expressed as the percentage of triglycerides, based on the quantity of total glycerides. The starting product Tonalin TG 80 has a triglyceride content of 81%, based on the quantity of total glycerides. Evaluation is based on the percentage area. The acid value of each sample was also measured.

TABLE 1
Enzyme screening for the selective hydrolysis
of diglycerides in CLA-TG

| Mix- | | | | | |
|---|---|---|---|---|---|
| ture | Enzyme | Manufacturer | Organism | Quantity | |
| 1 | Lipase A | Amano | Aspergillus nig. | 100 | mg |
| 2 | Novozym 735 | Novozymes | Candida ant. A | 100 | μl |
| 3 | Novozym 525 | Novozymes | Candida ant. B | 100 | μl |
| 4 | Lipomod 34 | Biocatalysts | Candida cyl. | 20 | mg |
| 5 | Lipase | Amano | Mucor | 50 | mg |
| 6 | Novozym 388 | Novozymes | Rhizomucor | 50 | μl |
| 7 | Lipase G | Amano | Penicilium cam. | 20 | mg |
| 8 | Lipase R | Amano | Penicilium roq. | 50 | mg |
| 9 | Lipase L115 | Biocatalysts | Porcine pancreas | 50 | mg |
| 10 | Lipase PS | Amano | Pseudomonas | 20 | mg |
| 11 | Lipomod 36 | Biocatalysts | Rhizopus | 20 | mg |
| 12 | Lipase FAP-15 | Amano | Rhizopus | 20 | mg |
| 13 | Lipozym TL 100 | Novozymes | Thermomyces | 20 | μl |

| | | | | % TG/ |
|---|---|---|---|---|
| Mix- | | % TG/total TG | | total TG |
| ture | AV (1.5 h) | (1.5 h) | AV (5 h) | (5 h) |
| 1 | 32 | 74 | 57 | 60 |
| 2 | 43 | 60 | 46 | 60 |
| 3 | 14 | 94 | 13 | 98 |
| 4 | 119 | 22 | 129 | 22 |
| 5 | 18 | 79 | 16 | 82 |
| 6 | 18 | 87 | 18 | 87 |
| 7 | 41 | 91 | 44 | 94 |
| 8 | 7 | 86 | 10 | 82 |
| 9 | 2 | 85 | 3 | 87 |
| 10 | 80 | 34 | 86 | 36 |
| 11 | 66 | 37 | 65 | 41 |
| 12 | 71 | 45 | 71 | 29 |
| 13 | 9 | 87 | 16 | 88 |

Result:

It is clear from the enzyme screening that Candida antarctica B lipase (Novozym 525) and Penicilium camembertii lipase (Lipase G) significantly increase the triglyceride content in commercially available Tonalin TG 80 from 81% to 98% or 94%. A slight increase in the triglyceride content was detected in the case of the lipases Novozym 388, Lipase R, Lipase L115 and Lipozym TL 100.

### Example 2

### Enrichment of Triglyceride as a Function of the Enzyme Concentration of Novozym 525 and Lipase G and as a Function of the Quantity of Water used

6 reaction vessels were each filled with 20 g Tonalin TG 80. Water and enzyme, as indicated in Table 2 below, were then added to vessel. The mixtures were incubated with stirring at room temperature. Samples were taken after 1 h, 5 h and 22 h, the oil phase was removed by centrifuging and the distribution of the glycerides was analyzed by gas chromatography. The result is expressed as the ratio of triglycerides, based on the quantity of total glycerides. The starting product Tonalin TG 80 has a triglyceride content of 81%, based on the quantity of total glycerides. Evaluation is based on the percentage area. The acid value of each sample was also measured.

TABLE 2
Enrichment of triglyceride as a function of the enzyme
concentration of Novozym 525 and lipase G and as a
function of the quantity of water used

| Mix- | | | AV | |
|---|---|---|---|---|
| ture | Water | Enzyme | (1 h) | % TG/total TG (1 h) |
| 1 | 0.5 g | 50 μl Novozym 525 | 17 | 96 |
| 2 | 0.5 g | 100 μl Novozym 525 | 18 | 98 |
| 3 | 1 g | 100 μl Novozym 525 | 25 | 96 |
| 4 | 0.5 g | 2 mg Lipase G | 15 | 88 |
| 5 | 0.5 g | 5 mg Lipase G | 15 | 90 |
| 6 | 1 g | 5 mg Lipase G | 30 | 93 |
| Mix- | AV | | AV | |
| ture | (5 h) | % TG/total TG (5 h) | (22 h) | % TG/total TG (22 h) |
| 1 | 18 | 97 | 17 | 98 |
| 2 | 16 | 99 | 17 | 99 |
| 3 | 23 | 96 | 23 | 95 |
| 4 | 24 | 88 | 19 | 89 |
| 5 | 23 | 92 | 21 | 96 |
| 6 | 36 | 94 | n.d. | 97 |

Result:

A distinct enrichment of the triglyceride content was achieved in every case highest triglyceride content was achieved with Novozym 525 for a water content of 2.5% and an enzyme concentration of 0.5%.

### Example 3

### Synthesis of CLA Triglyceride Coupled With Selective Hydrolysis of the Partial Glycerides

85 g CLA free acid, 9 g glycerol, 6 g immobilized Candida antarctica B lipase and 0.2 g sodium carbonate dissolved in 0.4 g water were introduced into a flask. The mixture was incubated with stirring at a temperature of 70° C. and in a vacuum of 60 mbar. After 24 h, the vacuum was removed and a sample was taken for analysis by gas chromatography. The temperature was lowered to 45° C. and 20 g water were added. The mixture was incubated for 2 h, samples being taken after 1 h and 2 h for analysis by gas chromatography. The water phase was then separated from the oil phase by centrifuging at 12,000 r.p.m. After the separation, the glyceride distribution of the oil phase was analyzed by gas chromatography.

TABLE 3
Synthesis of CLA-TG coupled with selective
hydrolysis of the partial glycerides

| | | | | Triglyc- | |
|---|---|---|---|---|---|
| | Fatty acid | Monoglyc. | Diglyceride | eride | % Triglyc./ |
| Sample | [%] | [%] | [%] | [%] | total glyc. |
| Starting | 100 | 0 | 0 | 0 | 0 |
| mixture | | | | | |
| 24 h | 9.2 | 0 | 20.8 | 70.0 | 77 |
| synthesis | | | | | |
| 1 h | 27.8 | 0 | 4.5 | 67.7 | 94 |
| hydrolysis | | | | | |
| 2 h | 28.3 | 0 | 3.2 | 68.5 | 96 |
| hydrolysis | | | | | |
| After | 28.6 | 0 | 2.9 | 68.5 | 96 |
| separation | | | | | |

Result:

The enzymatic synthesis of CLA triglyceride can be directly combined with the selective back-hydrolysis of the partial glycerides in a one-pot reaction providing Candida antarctica B lipase is used. The coupled synthesis/hydrolysis reaction leads to triglyceride contents of >95% in a distinctly faster time than the direct synthesis.

### Example 4

### Synthesis of Medium-Chain Triglyceride (MCT) Coupled With Selective Hydrolysis of the Partial Glycerides

100 g caprylic acid, 19 g glycerol, 5 g immobilized Candida antarctica B lipase and 0.2 g sodium carbonate dissolved in 0.4 g water were introduced into a flask. The mixture was incubated with stirring under nitrogen at a temperature of 70° C. and in a vacuum of 60 mbar. After 45 h, the vacuum was removed and a sample was taken for analysis by gas chromatography. The immobilized enzyme was filtered off. Quantities of 10 g of the MCT mixture and 0.5 g water were weighed into 2 vessels. 10 μl Novozym 525 were then added to mixture 1 and 1 mg Lipase G to mixture 2. The two mixtures were incubated with stirring for 24 h at room temperature. Samples were taken after 1 h, 5 h and 24 h for analysis of the glyceride distribution by gas chromatography.

TABLE 4
Synthesis of medium chain triglyceride (MCT) coupled
with selective hydrolysis of the partial glycerides

| | Fatty | | Diglyc- | Triglyc- | |
|---|---|---|---|---|---|
| | acid | Monoglyc. | eride | eride | % Triglyc./ |
| Sample | [%] | [%] | [%] | [%] | total glyc. |
| Starting mixture | 100 | 0 | 0 | 0 | 0 |
| 45 h synthesis | 15.2 | 0 | 4.2 | 80.5 | 95 |
| Mixture 1 | | | | | |
| 1 h hydrolysis | 17.3 | 0.4 | 1.5 | 80.8 | 97.7 |
| 2 h hydrolysis | 17.4 | 0.3 | 1.4 | 80.9 | 97.9 |
| 24 h hydrolysis | 17.5 | 0.3 | 1.3 | 80.9 | 98.1 |
| Mixture 2 | | | | | |
| 1 h hydrolysis | 17.3 | 0.3 | 1.8 | 80.6 | 97.5 |
| 2 h hydrolysis | 17.4 | 0.4 | 1.7 | 80.5 | 97.4 |
| 24 h hydrolysis | 18.0 | 0.3 | 1.1 | 80.6 | 98.3 |

Result:

An increase in the triglyceride content is achieved both with Novozym 525 and with Lipase G.

### Example 5

### Synthesis of Medium Chain Triglyceride (MCT) Coupled With Selective Hydrolysis of the Partial Glycerides and Purification of the MCT by Distillation

1050 g caprylic acid, 200 g glycerol, 50 g immobilized Candida antarctica B lipase and 2 g sodium carbonate dissolved in 5 g water were introduced into a flask. The mixture was incubated with stirring under nitrogen at a temperature of 60° C. and in a vacuum of 5 mbar. After 24 h, the vacuum was removed and a sample was taken for analysis by gas chromatography. The immobilized enzyme was filtered off. 200 g water and 7 ml Candida antarctica B lipase in a liquid formulation were added to the mixture which was then incubated with stirring for 2 h at room temperature. After 2 h, the pH was adjusted to 3.0 with 0.1 M HCI and the water phase was separated by centrifuging. The oil phase was washed with 200 g water and the aqueous phase was again separated by centrifuging. Caprylic acid and monoglycerides were then removed by distillation at 190° C. under a vacuum of 1 mbar using a thin-layer evaporator. The bottom product was filtered and subjected to analysis. The bottom product was then bleached with bleaching earth and active carbon and refined for the same time with concentrated sodium hydroxide.

TABLE 5
Synthesis of medium chain triglyceride (MCT) coupled
with selective hydrolysis of the partial glycerides and
purification of the MCT by distillation

| | Fatty | | | | |
|---|---|---|---|---|---|
| | acid | Monoglyc. | Diglyc. | Triglyc. | % TG/ |
| Sample | [%] | [%] | [%] | [%] | total glyc. |
| Starting mixture | 100 | 0 | 0 | 0 | 0 |
| 6 h synthesis | 32.3 | 1.4 | 34.0 | 32.1 | 47.6 |
| 24 h synthesis | 14.3 | 0 | 3.3 | 82.3 | 96.1 |
| 2 h back-hydrolysis | 14.8 | 0.2 | 0.9 | 84.2 | 98.7 |
| Dist. bottom product | 0.4 | 0 | 0.9 | 98.6 | 99.1 |
| Sample after refining | 0 | 0 | 1.2 | 98.8 | 98.9 |

Lovibond 1 color

| | Acid value | OH value | Yellow | Red |
|---|---|---|---|---|
| Dist. bottom product | 0.8 | | 0.7 | 0.4 |
| Sample after refining | 0.2 | 1.3 | 0.2 | 0.1 |

Result:

A very high triglyceride content can be achieved by enzymatic back-20 hydrolysis coupled with distillation and refining, so that the low OH values cosmetic MCT products are expected to show are also achieved.

1. A process for the enzyme-catalyzed synthesis of a member selected from the group consisting of esters of polyunsaturated fatty acids, esters of short-chain fatty acids, esters of sensitive fatty acids and mixtures thereof with a high triglyceride content, which comprises:
(a) reacting fatty acids or alkyl esters thereof with glycerol to form their triglycerides in vacuo in the presence of an enzyme under low-water conditions to form a first reaction mixture,
(b) selectively, back hydrolyzing partial glycerides formed in step (a) in the first reaction mixture in the presence of an enzyme after the addition of water to form a second reaction mixture, and
(c) separating water, fatty acids or alkyl esters and glycerol from the second reaction mixture to form a composition with a high triglyceride content.

2. The process as claimed in claim 1, wherein, step (a) and step (b) are carried out in the same reactor.

3. The process as claimed in claim 1, wherein, steps (a) and (b) are carried out in the presence of the same enzyme.

4. The process as claimed in claim 1, wherein, the fatty acids and alkyl esters thereof are selected from the group consisting of short-chain fatty acids with 6 to 12 carbon atoms, esters of short-chain fatty acids with 6 to 12 carbon atoms with C₁₋₂ alkyl alcohol, docosahexaenoic acid, docosahexaenoic acid esters with C₁₋₂ alcohols, eicosapentaenoic acid, eicosapentaenoic acid esters with C₁₋₂ alcohols, linoleic acid, linoleic acid esters with C₁₋₂ alcohols, conjugated linoleic acid , conjugated linoleic acid esters with C₁₋₂ alcohols, and mixtures thereof.

5. The process as claimed in claim 1, wherein, the enzyme comprises at least one member selected from the group consisting of lipases, phospholipases, and esterases.

6. The process as claimed in claim 1, wherein, the enzymes are immobilized and/or in a chemically modified form.

7. The process as claimed in claim 1, wherein, Candida antarctica B lipase is used as the enzyme in both steps (a) and (b).

8. The process as claimed in claims 1, wherein, step (a) is carried out under a vacuum of at least 200 mbar step (b) is carried out under normal pressure.

9. The process as claimed in claim 1, wherein, step (b) is carried out with from 1% to 50% water in the reaction mixture, based on the mixture as a whole.

10. Fatty acid glyceride esters obtained by the process claimed in claim 1.

11. Fatty acid esters of polyunsaturated fatty acids with a triglyceride content of at least 90% by weight.

12. Fatty acid esters of conjugated linoleic acid with a triglyceride content of at least 90% by weight.

13. Fatty acid esters of medium chain fatty acids with a triglyceride content of at least 90% by weight.

14. Fatty acid esters consisting of mixtures of esters of at least two members selected from the group consisting of medium chain fatty acids, conjugated linoleic acid and polyunsaturated fatty acids with a triglyceride content of at least 90% by weight.

15. The process of claim 1, wherein, step (a) is carried out at a temperature of from 40° C. to 90° C. at a pressure of from 1 to 100 mbar and step (b) is carried out at a temperature of from 20° C. to 60° C.

16. The process of claim 15, wherein, step (a) is carried out at a temperature of 60° C. to 80° C. and a pressure of from 5 to 60 mbar and step (b) is carried out at a temperature of from 20° C. to 45° C. and a water concentration of from 2% to 25% by weight of the second mixture.

17. The process of claim 1, wherein, at least one alkaline salt is added to a reaction mixture of claim 1 in a dry form or in admixture with water.

18. The process of claim 1, wherein, the water is removed from the second reaction mixture by a process selected from the group consisting of centrifugation and distillation optionally after acidification.

19. The process of claim 18, wherein, the esters with a triglyceride content of at least 90% by weight of the ester is recovered from the second reaction mixture by a process selected from the group consisting of distillation and a combination of centrifugation and distillation.
